(19) United States
(12) Patent Application Publication
(10) Pub. No.: US 2023/0375858 A1
(43) Pub. Date: Nov. 23, 2023

(54) CONTACT LENS IN WHICH BASICALLY CENTRIC PART COMPRISES EMPTY SPACE, AND METHOD FOR WEARING THE SAME

(71) Applicant: WellC, Busan (KR)

(72) Inventor: KI SUNG PARK, Hwaseong-si (KR)

(73) Assignee: WellC, Busan (KR)

(21) Appl. No.: 17/885,314

(22) Filed: Aug. 10, 2022

(30) Foreign Application Priority Data
May 19, 2022 (KR) 10-2022-0061187

(51) Int. Cl. G02C 7/04

(52) U.S. Cl. CPC G02C 7/049

(57) ABSTRACT

A contact lens and method for wearing the contact lens is provided. Although there is no vision correction function, contact lenses with the function of treating dry eyeball are provided. The contact lens comprises a centric part formed of an empty space, and a surrounding part comprising a transparent material through which light is transmitted. Therefore, when the lens is worn on an user's eyeball, a centric area of the eyeball is exposed to the air and has the function of storing and maintaining tears.

### TECHNICAL FIELD

The present invention relates to a contact lens in which basically centric part comprises an empty space, and a method for wearing the same.

### BACKGROUND ART

Since the cornea has a gel-type material, ectasia due to intraocular pressure occurs in the thin portion of the cornea. In this case, the dilated portion creates a curvature deviation, which causes abnormal vision and also causes a tear film defect, thereby resulting in xerophthalmia. If corneal ectasia due to intraocular pressure continues, eventually, a situation in which the cornea is perforated may occur.

Existing dry eye treatment mainly uses a method of supplying tears (water, mucus oil), but even if tears are supplied, tears in the protruding area produced by corneal deformation cannot be maintained and flow quickly, so dry eye syndrome cannot be solved.

In addition, existing contact lenses are used for vision correction or beauty, and soft lenses absorb water, which can cause dry eyes.

Therefore, so far, a method of perforations around the lens for only tear circulation has been executed, but the outcome has been insignificant.

### SUMMARY

Aspects of the present invention is to provide a contact lens in which basically centric part comprises an empty space.

Another aspects of the present invention is to provide a method for wearing a contact lens in which basically centric part comprises an empty space.

According to some aspects of the present disclosure, A contact lens comprises a cover part that is worn on an eye and comprising a transparent material through which light is transmitted, a centric part located at the center of the cover part and storing tears on an eyeball, and exposing a surface of the eyeball.

According to some aspects, the contact lens further comprises a skeleton part that comprises a ring assembly comprising a plurality of rings having the same center and a plurality of connection members connecting the plurality of rings, wherein the cover part has a form in which the skeleton part can be inserted, and the centric part is empty, wherein the cover part comprises a plurality of protrusions formed on one surface of the cover part.

According to some aspects, the one surface of the cover part is a surface relatively close to a user's eyeball.

According to some aspects, the plurality of protrusions is regularly arranged in a predetermined pattern, or randomly arranged on the one surface of the cover part.

According to some aspects, the cover part has a toric form of a predetermined size.

According to some aspects, a material of the cover part is at least one of silicone hydrogel, HAMA hydrogel, Rigid Gas Permeable (RGP), polymethyl methacrylate (PMMA), Polyvinyl Alcohol (PVA), hypergel, and cellulose acetate butylate (CAB).

According to some aspects, the plurality of rings has shapes having concentric circle shapes having different sizes, or ellipse shapes having the same eccentricity and different sizes from each other.

According to some aspects, each of the plurality of connection members is disposed to be spaced apart from an adjacent connection member by the same angle with respect to a center of the ring assembly.

According to some aspects, the contact lens further comprises an auxiliary connection member connecting between the plurality of rings and disposed where the connection member is not disposed.

According to some aspects, a material of the ring is at least one of polymethyleth acrylate (PMMA), gold, alloy, platinum, titanium, and stainless steel.

According to some aspects of the present disclosure, a method for wearing a contact lens comprises a step of selecting a contact lens to be worn on the user's eyeball and a step of wearing the selected contact lens to the user's eyeball, wherein the contact lens includes a skeleton part that includes a ring assembly including a plurality of rings having the same center and a plurality of connection members connecting the plurality of rings and a cover part into which the skeleton part is configured to be inserted and which has a form a centric part thereof is empty.

According to some aspects, the step of selecting the contact lens to be inserted includes a step of grasping a corneal state of a portion where the contact lens is to be worn, and a step of selecting a contact lens corresponding to the grasped corneal state among a contact lens set.

According to some aspects, the corneal state includes at least one of a size, a curvature, and a moisture content of a user's cornea.

According to some aspects, in each contact lens of the contact lens set, at least one of a protrusion height of a plurality of protrusions formed on one surface of the cover part, an interval between the protrusions, a material of the contact lens, and a thickness of the contact lens is different from each other.

According to some aspects, in each contact lens of the contact lens set, at least one of a curvature, a distance from a center of the ring assembly to a ring having the smallest size among the plurality of rings, a distance from the center of the ring assembly to a ring having the largest size among the plurality of rings, an interval between the plurality of rings, and whether the interval between the plurality of rings is constant is different from each other.

According to some aspects, the method for wearing a contact lens further comprises a step of selecting again any one contact lens among the contact lens set based on a user's wearing feedback for the selected contact lens and the grasped corneal state.

According to some aspects, the step of wearing the selected contact lens to the user's eyeball comprises a step of generating an incised portion by incising a part of the user's cornea, a step of irradiating light through the incised portion and a step of wearing the contact lens to cover a part of an upper region of the incised portion.

According to some aspects, the contact lens further comprises a plurality of protrusions formed on one surface of the cover part.

According to some aspects, the one surface of the cover part is a surface relatively close to the user's eyeball.

According to some aspects, the plurality of protrusions is regularly arranged in a predetermined pattern, or randomly arranged on the one surface of the cover part.

According to some aspects of the present disclosure, a contact lens comprises a centric part and an eccentric part, wherein the centric part is a region to a circle or an ellipse having a predetermined distance from a central point of the contact lens, the eccentric part is a region excluding the central region in the contact lens, the center part basically includes an empty space, and the eccentric part optionally includes an empty space in some cases.

According to some aspects, the contact lens further comprises a plurality of protrusions formed on one surface of the contact lens.

According to some aspects of the present disclosure, A skeleton part comprises a ring assembly comprising a plurality of rings having the same center, wherein the plurality of rings have the shape of concentric circles with different sizes, and a plurality of connection members for connecting between the plurality of rings, wherein each of the plurality of connection members is disposed apart from an adjacent connection member by the same angle with respect to the center.

According to some aspects of the present disclosure, A skeleton part comprises a ring assembly comprising a plurality of rings having the same center, wherein the plurality of rings have the shape of concentric circles with different sizes, a plurality of connection members for connecting between the plurality of rings, wherein each of the plurality of connection members is disposed apart from an adjacent connection member by the same angle with respect to the center, and an auxiliary connection member connecting between the plurality of rings and disposed where the connection member is not disposed.

According to some aspects of the present disclosure, A skeleton part comprises a ring assembly comprising a plurality of rings having the same center, wherein the plurality of rings have the shape of concentric circles with different sizes, and a plurality of connection members for connecting between the plurality of rings, wherein each of the plurality of connection members is disposed apart from an adjacent connection member by the same angle with respect to the center, wherein the material of the ring is at least one of polymethylmethacrylate (PMMA), gold, alloy, platinum, titanium, and stainless steel.

According to some aspects of the present disclosure, A skeleton part comprises a ring assembly comprising a plurality of rings having the same center, wherein the plurality of rings have the shape of ellipses with the same eccentricity and different sizes, and a plurality of connection members for connecting between the plurality of rings, wherein each of the plurality of connection members is disposed apart from an adjacent connection member by the same angle with respect to the center.

According to some aspects of the present disclosure, A skeleton part comprises a ring assembly comprising a plurality of rings having the same center, wherein the plurality of rings have the shape of ellipses with the same eccentricity and different sizes, a plurality of connection members for connecting between the plurality of rings, wherein each of the plurality of connection members is disposed apart from an adjacent connection member by the same angle with respect to the center, and an auxiliary connection member connecting between the plurality of rings and disposed where the connection member is not disposed.

Aspects of the present disclosure are not limited to those mentioned above, and other objects and advantages of the present disclosure that have not been mentioned can be understood by the following description, and will be more clearly understood by embodiments of the present disclosure. In addition, it will be readily understood that the objects and advantages of the present disclosure can be realized by the means and combinations thereof set forth in the claims.

The xerophthalmia can be eliminated and corneal ectasia due to the intraocular pressure can also be prevented by wearing the contact lens including the empty space in the centric part, on the cornea.

In addition, dryness of tears of the cornea can be eliminated and corneal deformation in the corneal periphery can be corrected with the contact lens including the empty space in the centric part.

That is, by providing the empty space in the centric part basically and the eccentric part optionally includes an empty space in some cases, dryness of tears of the cornea can be eliminated, and the shape correction of the corneal periphery can be supplemented by inserting the skeletal part into the cover part to be worn on the corneal periphery.

The effect of the shape correction of the cornea can be further maximized by continuously wearing the contact lens of the present invention in daily life rather than wearing the contact lens only during a specific time period (for example: sleeping time).

The specific effects of the present invention in addition to the above will be described together while explaining the specific details for carrying out the invention below.

### BRIEF DESCRIPTION OF THE DRAWINGS

FIGS. 1A to 1D are schematic diagrams of constituent tissues of a cornea and diagrams for explaining a process of corneal ectasia due to an intraocular pressure.

FIG. 2 is a view showing a member for intraocular insertion for preventing corneal ectasia due to the intraocular pressure according to some embodiments inserted into the eyeball.

FIG. 3 is a view showing a skeleton part and a cover part of the contact lens according to some embodiments.

FIGS. 4A and 4B are views showing a cover part according to some embodiments.

FIGS. 5A and 5B are views showing a cover part according to some other embodiments.

FIG. 6 is a view for explaining a skeleton part according to some embodiments.

FIG. 7 is a flowchart showing a method for wearing the contact lens according to some embodiments.

FIGS. 8A and 8B are flowcharts showing a method for wearing the contact lens according to some embodiments.

FIGS. 9A to 9F are flowcharts showing a method for wearing the contact lens according to some embodiments and schematic diagrams for explaining the same.

### DETAILED DESCRIPTION OF THE PREFERRED EMBODIMENTS

The terms or words used in the present disclosure and the claims should not be construed as limited to their ordinary or lexical meanings. They should be construed as the meaning and concept in line with the technical idea of the present disclosure based on the principle that the inventor can define the concept of terms or words in order to describe his/her own embodiments in the best possible way. Further, since the embodiment described herein and the configurations illustrated in the drawings are merely one embodiment in which the present disclosure is realized and do not represent all the technical ideas of the present disclosure, it should be understood that there may be various equivalents, variations, and applicable examples that can replace them at the time of filing this application.

Although terms such as first, second, A, B, etc. used in the present description and the claims may be used to describe various components, the components should not be limited by these terms. These terms are used only for the purpose of distinguishing one component from another. For example, a first component may be referred to as a second component, and similarly, a second component may be referred to as a first component, without departing from the scope of the present disclosure. The term ‘and/or’ includes a combination of a plurality of related listed items or any item of the plurality of related listed items.

The terms used in the present description and the claims are merely used to describe particular embodiments and are not intended to limit the present disclosure. Singular expressions include plural expressions unless the context explicitly indicates otherwise. In the present application, terms such as “comprise”, “have”, “include”, “contain”, etc. should be understood as not precluding the possibility of existence or addition of features, numbers, steps, operations, components, parts, or combinations thereof described herein.

When a part is said to include “at least one of a, b or c”, this means that the part may include only a, only b, only c, both a and b, both a and c, both b and c, all of a, b and c, or variations thereof.

Unless otherwise defined, all terms used herein, including technical or scientific terms, have the same meaning as commonly understood by those of ordinary skill in the art to which the present disclosure pertains.

Terms such as those defined in commonly used dictionaries should be construed as having a meaning consistent with the meaning in the context of the relevant art, and are not to be construed in an ideal or excessively formal sense unless explicitly defined in the present disclosure.

In addition, each configuration, procedure, process, method, or the like included in each embodiment of the present disclosure may be shared to the extent that they are not technically contradictory to each other.

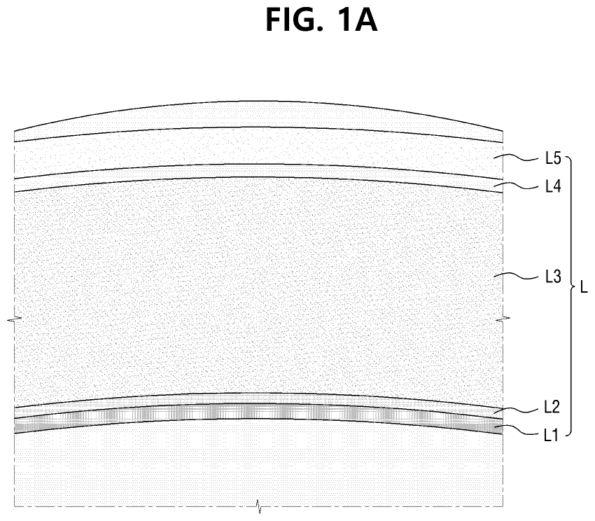

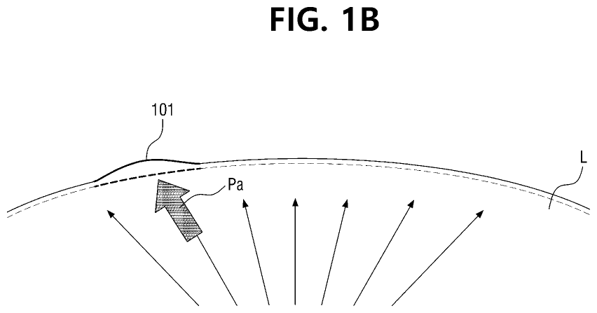

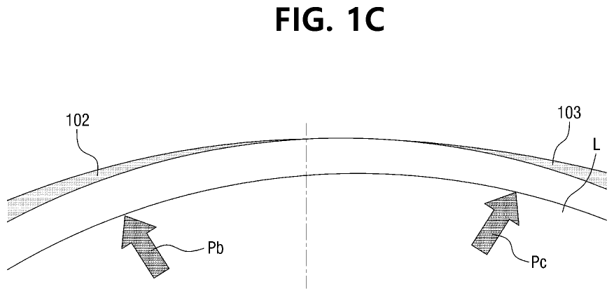

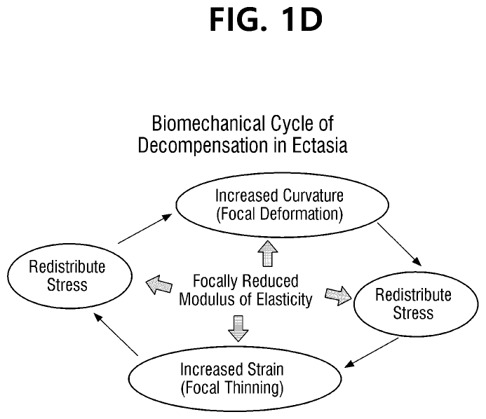

Hereinafter, a contact lens and a method for wearing the contact lens according to some embodiments of the present invention will be described with reference to FIGS. 1A to 12F.

FIGS. 1A to 1D are schematic diagrams of constitutive tissues of the cornea and drawings for explaining the process of corneal ectasia due to intraocular pressure.

FIG. 1A shows a schematic diagram of constitutive tissues of the cornea. Referring to FIG. 1A, the cornea L of the eye may be divided into five layers. In this case, L1 may represent the corneal endothelium, L2 may represent the posterior border plate (descemet's membrane), L3 may represent the corneal stroma, L4 may represent Bowman's membrane, and L5 may represent the corneal epithelium. However, the cornea of the eye is not limited thereto, and may be divided into more subdivided layers, and some layers may be omitted and called while being merged with other layers.

In general, the thickness of the corneal epithelium L5 is about 50 microns, and the thickness of the corneal stroma L3 is 300 microns or more and 800 microns or less. In this case, if the thickness of the entire cornea L is 360-400 microns or less, it may be seen that the probability of showing keratectasia is high. However, if the intraocular pressure is high, keratectasia may also occur even if the cornea is thick.

FIGS. 1B to 1D are drawings for explaining the process of corneal ectasia due to intraocular pressure.

A thin portion of the cornea of the eye may be dilated by intraocular pressure. For example, FIG. 1B shows a portion 101 in which the cornea L is dilated in the direction of the arrow Pa due to intraocular pressure, and FIG. 1C shows portions 102 and 103 in which the cornea L is dilated in the directions of arrows Pb and Pc due to intraocular pressure.

In this case, the dilated portion may create a curvature deviation, which causes abnormal vision and also causes a tear film defect, thereby resulting in xerophthalmia. If the corneal ectasia due to intraocular pressure continues, eventually, the cornea may also be perforated.

FIG. 1D shows the biomechanical cycle of decompensation in corneal ectasia. The cycle shown in FIG. 1D means that if it is initiated by the asymmetry of the distribution of biomechanical properties, then the cornea becomes thinner and the stress increases, and eventually the cornea deforms or redistributes its curvature in a compensatory manner.

Therefore, it is necessary to insert a biocompatible material into the cornea to prevent deformation or ectasia of the cornea due to intraocular pressure, and hereinafter, a member for intraocular insertion that performs such a function will be described in detail.

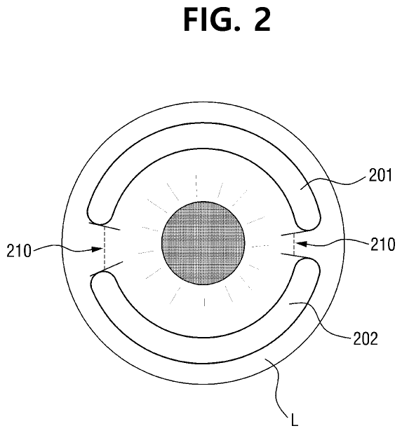

FIG. 2 shows the member for intraocular insertion according to some embodiments inserted into the eye.

Referring to FIG. 2, FIG. 2 shows the upper region 201, the lower region 202, and the empty space 210 between the upper and lower regions, of the member for intraocular insertion inserted into the cornea L of the eye. In this case, each of the upper region 201 and the lower region 202 of the member for intraocular insertion may have a semicircular shape.

In general, when this member for intraocular insertion is inserted into the eye, a tunnel is made in the corneal stroma portion (L3 in FIG. 1A), and then the upper region 201 and the lower region 202 of the member for intraocular insertion are inserted axially symmetrically. In this case, by using the principle that the central cornea is flattened by generating an arc-shortening effect of the cornea, the progression of keratoconus may be suppressed.

However, this member for intraocular insertion may have side effects in that the cornea at the site into which the ring piece is inserted is protruded, and due to this, irregular refractive power and tear film defects are induced. In addition, since the upper region 201 and the lower region 202 of the member for intraocular insertion are separated and inserted axially symmetrically, the curvature of the portions 201 and 202 in which each region is disposed and the curvature of the portion 210 in which each region is not disposed may be different. Therefore, in this case, it may be difficult to evenly improve the shape of the cornea to have a spherical or elliptical curvature.

Therefore, the shape of the contact lens capable of solving these problems and the effect thereof will be described below.

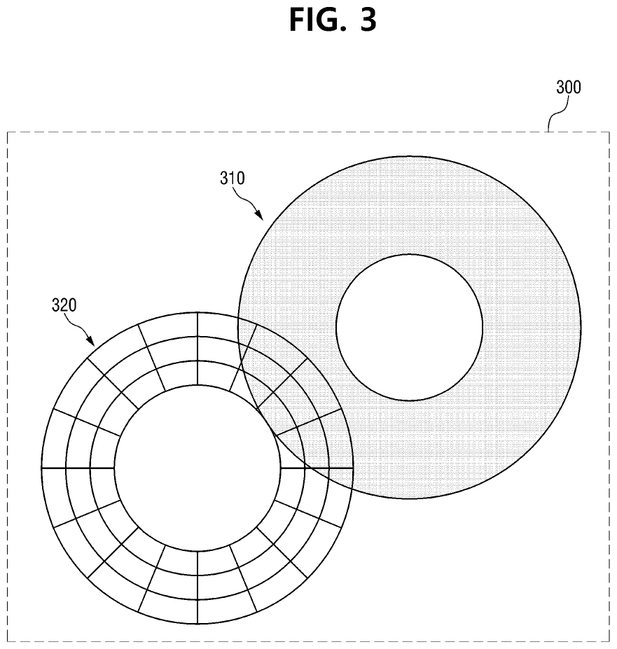

FIG. 3 shows a skeleton part and a cover part of the contact lens including an empty space in the centric part basically and the eccentric part optionally includes an empty space in some cases, according to some embodiments.

Referring to FIG. 3, the contact lens 300 including an empty space in the centric part according to some embodiments may include a cover part 310 and a skeleton part 320.

The cover part 310 can be worn on the eye and includes a transparent material through which light is transmitted.

In some examples, the material of the cover part 310 may be formed of a material such as polymethyleth acrylate (PMMA), gold, alloy, platinum, titanium, and stainless steel, but embodiments are not limited thereto.

The cover part comprises a penetration hole for storing tears on the cornea. In this case, the penetration hole may serve to store tears on the cornea.

The penetration hole can be formed in the centric part of the cover part or can be formed in the eccentric part of the cover part.

In some examples, the cover part 310 may have a shape with an empty centric part. The centric part may mean a region spaced apart by the same predetermined distance from the central point of the cover part 310. In this case, the shape of the empty centric part may be circular as shown in FIG. 3, but is not limited thereto. In some examples, the cover part 310 may have a toric or ellipsoidal toric as shown in FIG. 3, but is not limited thereto.

In some other examples, the cover part 310 may have a shape in which a part of the eccentric part is empty. The eccentric part may be a region other than the centric part of the cover part 310. For example, any one region of the eccentric part may be formed as an empty space. The shape of the empty space of the eccentric part may be circular, but is not limited thereto.

As some other examples, the cover part 310 may have a shape in which the centric part is empty while at the same time a portion of the eccentric part is empty. However, it is not limited to the above example.

The thickness of the cover part 310 may be, for example, 0.5 mm or more and 20 mm or less, but is not limited thereto.

The skeleton part 320 may be inserted into the cover part 310. In FIG. 3, the cover part 310 and the skeleton part 320 are shown in a form in which some regions overlap, but this is only for convenience of description.

The cover part 310 may be formed in a shape into which the skeleton part 320 can be inserted.

In some examples, the cover part 310 may be formed with the same or a larger size (for example: diameter, thickness, or the like) and/or the same or similar curvature as that of the skeleton part 320 so that the skeleton part 320 can be inserted.

The skeleton part 320 may include a ring assembly and a plurality of connection members.

The ring assembly may include a plurality of rings having the same center. The plurality of connection members may connect between the plurality of rings.

The thickness of the skeleton part 320 may be, for example, 100 microns or less, but is not limited thereto.

The material of the cover part 310 and/or the skeleton part 320 may be a biocompatible material. In this case, the biocompatible material may be a product made of or coated with a material harmless to the human body.

In some examples, the material of the skeleton part 320 may be formed of any one of a material such as silicone hydrogel, HAMA hydrogel, Rigid Gas Permeable (RGP), polymethyl methacrylate (PMMA), Polyvinyl Alcohol (PVA), hypergel, cellulose acetate butylate (CAB), or a combination thereof. But embodiments are not limited thereto.

In some examples, unlike shown in FIG. 3, in the contact lens 300 including the empty space in the centric part basically and the eccentric part optionally includes an empty space in some cases, the skeleton part 320 may be omitted.

The contact lens 300 including the empty space in the centric part basically and the eccentric part optionally includes an empty space in some cases that has effects of being able to perform roles to prevent and treat xerophthalmia, defend against antigens that enter the cornea through external air and cause allergies or inflammation, fix the corneal flap (upper cornea) after eyeball surgery (for example: LASIK surgery), or the like.

The contact lens 300 may also be colored lenses. In this case, the contact lens may be in the form of a continuously wearable color contact lens that does not have a problem of oxygen permeability. In this case, the contact lens 300 has a has a new effect that it has a cosmetic use, there is no problem of oxygen permeability, and it can be worn continuously for a long time.

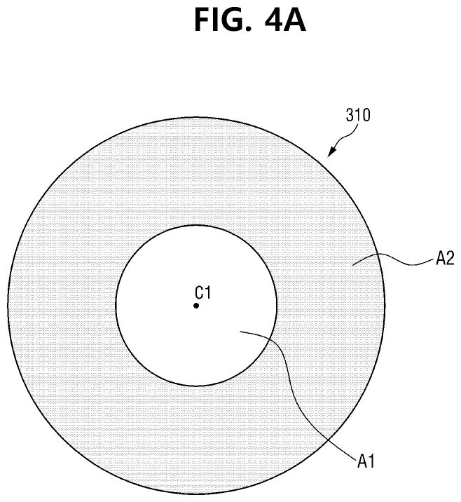

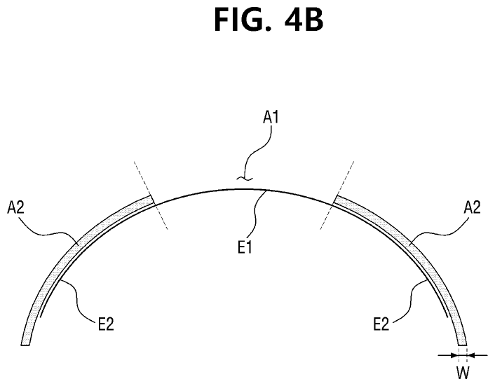

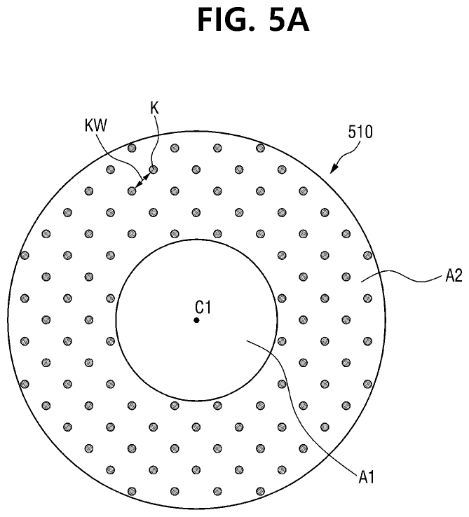

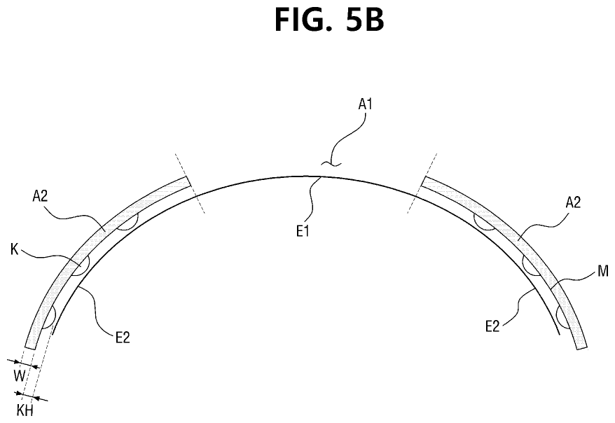

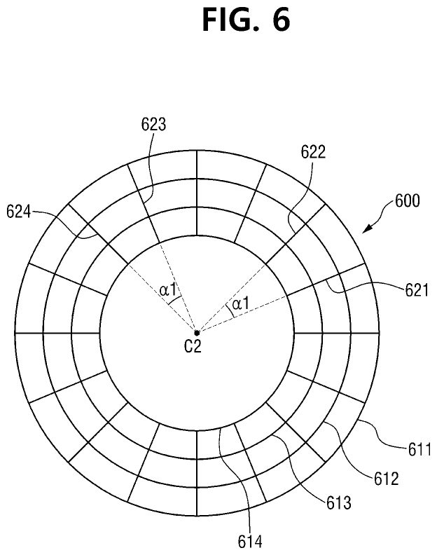

Hereinafter, the shape and form of the cover part 310 according to some embodiments will be described with reference to FIGS. 4A to 5B, and the shape and form of the skeleton part 320 according to some embodiments will be described with reference to FIG. 6.

FIGS. 4A and 4B show the cover part according to some embodiments.

Referring to FIG. 4A, the cover part 310 may include a centric part A1 and an eccentric part A2 with respect to the central point C1. The skeleton part may be inserted into the eccentric part A2. FIG. 4B shows the eccentric part A2 of the cover part of the contact lens worn on the user's corneas E1, E2, and E3.

Referring to FIGS. 4A and 4B, as described above with reference to FIG. 3, the centric part A1 of the cover part 310 may have the empty shape. However, unlike shown in FIGS. 4A and 4B, in some examples, as described above in FIG. 3, the centric part A1 and the eccentric part A2 may all include empty spaces.

Since the centric part A1 of the cover part 310 has the empty shape, the corneal centric part E1 at a position corresponding to the centric part A1 does not come into contact with the contact lens. Accordingly, it is possible to prevent corneal opacity caused by adsorption of the inner surface of the contact lens and the surface of the corneal centric part E1.

In some examples, since the centric part A1 of the cover part 310 has an empty shape, it has an effect of maintaining moisture in the corneal centric part E1 at a position corresponding to the centric part A1 of the cover part 310. That is, the cover part 310 of the contact lens has a predetermined thickness W, and may serve as a dam for confinement of a predetermined amount of tears flowing from the corneal centric part E1 to the corneal periphery E2. Accordingly, it can increase the time the tears stay in the cornea centric part E1.

Additionally, when a general contact lens is worn on the eyeball, the contact lens absorbs more moisture from the eyeball as the material of the contact lens has a higher water content. However, since the centric part A1 of the cover part 310 has the empty shape, the corneal centric part E1 does not come into contact with the contact lens, so that it is possible to partially prevent the contact lens from absorbing moisture from the eyeball.

The above-described effects may be similarly applied/generated even in a case where the eccentric part A2 of the cover part 310 includes the empty space.

Although the cover part 310 is shown as having the toric shape in FIG. 4A, the shape of the cover part 310 is not limited thereto. In some examples, the cover part 310 may have a shape such as an elliptical toric or other hollow polygons, unlike shown in FIG. 4A.

As described above, the contact lens 300 may be a color lens, and in this case, the cover part 310 may include a color.

FIGS. 5A and 5B show a cover part according to some other embodiments.

FIG. 5A shows a central point C1 of a cover part 510, an empty center A1, an eccentric part A2, and a plurality of protrusions K formed at a predetermined distance KW therebetween on one surface of the cover part 510. FIG. 5B shows the eccentric part A2 of the cover part of the contact lens worn on the user's corneas E1, E2, and E3, and a plurality of protrusions K that are formed on the surface M relatively close the user's corneas E1, E2, and E3, and have a predetermined height KH.

The plurality of protrusions K may be regularly arranged in a predetermined pattern.

In some examples, the plurality of protrusions K may be formed with a predetermined interval (for example: KW) from the adjacent protrusions. In addition, as shown in FIG. 5A, the plurality of protrusions K may be formed on a virtual straight line that divides the cover part 510 at predetermined intervals in horizontal, vertical, diagonal, and the like directions, thereby forming a honeycomb-shaped embossing pattern as a whole. However, the shape and pattern of the plurality of protrusions K are not limited thereto and may be freely modified.

FIG. 5A shows that the protrusions K are formed with the predetermined interval KW in all regions of the eccentric part A2, but the present invention is not limited thereto, and the protrusions K may be formed in some regions of the eccentric part A2 (for example: the upper region, the lower region, and the like), and the formation position thereof can be freely modified.

However, the embodiments are not limited thereto, the plurality of protrusions may be randomly arranged on the one surface of the cover part. Referring to FIG. 5B, as described above with reference to FIGS. 4A and 4B, the centric part A1 of the cover part 510 has the empty shape and has the predetermined thickness W, and thereby a certain amount of tears flowing from the corneal centric part E1 to the corneal peripheral portion E2 can be trapped.

In some examples, since the contact lens includes the plurality of protrusions K, one surface M of the eccentric part A2 does not come into contact with the cornea, accordingly, the space between the plurality of protrusions K can serve as a pathway through which a part of the tears flows. In addition, since the protrusion K comes into contact with the cornea, the contact lens can be moved slightly rather than fixed in a specific position in daily life after being worn by the user, thereby preventing the tears of the contact surface between the cornea and the lens from drying out and further preventing the cornea dryness.

In some examples, in a case where the distance KW between the plurality of protrusions K is reduced to form the protrusions K more densely or the height KH of the protrusions K is reduced, it can have the effect of trapping tears in the corneal centric part E1 for a longer period of time.

FIG. 6 is a view for explaining a skeleton part according to some embodiments.

FIG. 6 shows a skeleton part 600 according to some embodiments. Referring to FIG. 6, the skeleton part 600 may include a ring assembly and a plurality of connection members (for example: 621 to 624).

The ring assembly may comprise a plurality of rings 611 to 614.

In this case, the plurality of rings may have a common center point C2. In this case, the plurality of rings 611 to 614 may have the shape of concentric circles with different sizes or ellipse shapes with different sizes. However, the rings are not limited thereto, and the center of each ring may be partially different.

FIG. 6 shows that the intervals between the rings 611 to 614 is the same, but are not limited thereto, and the intervals between the rings may be partially different.

A plurality of connection members (e.g., 621 to 624) may connect between the plurality of rings 611 to 614. According to some embodiments, each of the plurality of connection members may be disposed apart from the adjacent connection members by the same angle α1 with respect to the center point C2, but is not limited thereto.

Each connection member, e.g., 621, may be integrally formed to have a length from the smallest ring 614 to the largest ring 611, or may be formed as a set of detailed connection members having a length equal to the distance between each ring (between 611 and 612, between 612 and 613, and between 613 and 614).

Although FIG. 6 shows that the number of connection members is sixteen, which is only an exemplary value, and the number of connection members may be freely modified.

FIG. 6 shows that each connection member is in a straight line form connecting from the smallest ring 614 to the largest ring 611, but is not limited thereto.

As some embodiments, the connection member may also be in a curved form connecting between each ring (between 611 and 612, between 612 and 613, and between 613 and 614).

As some other embodiments, the connection member may be formed differently between each ring (between 611 and 612, between 612 and 613, and between 613 and 614). For example, the connection member may also be formed to include one straight line form between the ring 611 and the ring 612, two straight line forms between the ring 612 and the ring 613, and one straight line form between the ring 613 and the ring 614.

In some other embodiments, the connection member may also be formed in a zigzag manner to connect between each ring (between 611 and 612, between 612 and 613, and between 613 and 614).

However, the connection member is not limited to the above-described embodiments, and may connect between each ring in various manners.

The thickness of the skeleton part 600 may be, for example, 100 microns or less, but is not limited thereto.

The skeleton part 600 may further include auxiliary connection members disposed where the respective connection members 621 to 624 are not disposed.

The contact lens including the empty space in at least one of the centric part and the eccentric part may be formed in various shapes, forms, materials, and the like, and is not limited to those shown in FIGS. 3 to 8.

The contact lens set including the empty space in the centric part basically and the eccentric part optionally includes an empty space in some cases may include a plurality of contact lenses having different shapes, forms, and/or materials.

In some examples, the contact lens set including the empty space in at least one of the centric part and the eccentric part may include a plurality of contact lenses which are different in material of the contact lens and/or thickness of the contact lens (FIGS. 4B and 5B, and W) including the shape of the protrusion (for example: the protrusion height KH in FIG. 5B), the interval between the protrusions (KW in FIG. 5B), and the material of the skeleton part, and the material of the cover part.

In some other examples, the contact lens set may include a plurality of contact lenses having different positions in the cover part where the empty space is formed. For example, the contact lens set may include a contact lens in which the centric part has the empty shape, a contact lens in which the centric and eccentric part include the empty space, a contact lens in which the centric part has the empty shape while the eccentric part includes the empty space, and the like. In this case, the contact lens set may include a plurality of contact lenses having different positions of the empty space included in the eccentric part.

In some other examples, the contact lens set may include a plurality of contact lenses having different shapes or forms of the skeleton part.

For example, the contact lens set may include a plurality of contact lenses different from each other in a curvature of the cover part and/or the skeleton part, a distance from the center of the ring assembly to a ring having the smallest size among the plurality of rings, and a distance from the center of the ring assembly to a ring having the largest size among the plurality of rings, the interval between the plurality of rings, whether the interval between the plurality of rings is constant, and the presence or absence of the auxiliary connection member disposed where the connection member is not disposed.

However, the present invention is not limited the examples described above, and the contact lens set including the empty space in the centric part basically and the eccentric part optionally includes an empty space in some cases may include contact lenses having more various shapes and forms.

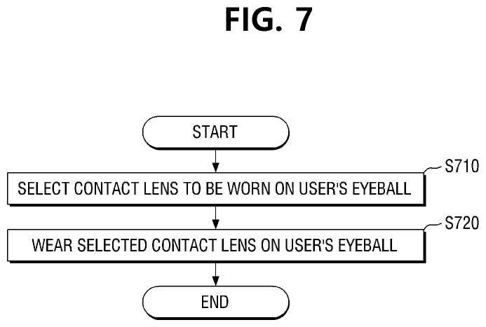

FIG. 7 is a flowchart showing a method for wearing a contact lens including the empty space in the centric part basically and the eccentric part optionally includes an empty space in some cases according to some embodiments.

First, the contact lens to be worn on the user's eyeball may be selected (S710).

In this case, any one of a plurality of contact lenses having different shapes, forms, and the like of the cover part and/or the skeleton part may be selected.

Then, the selected contact lens may be worn on the user's eyeball (S720).

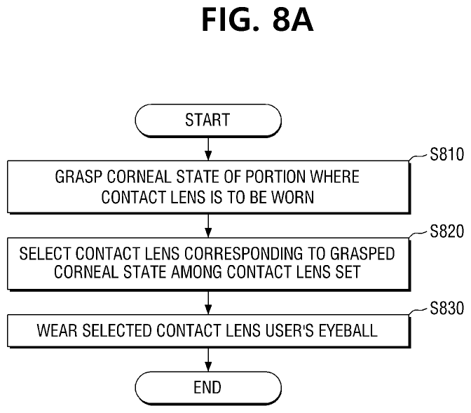

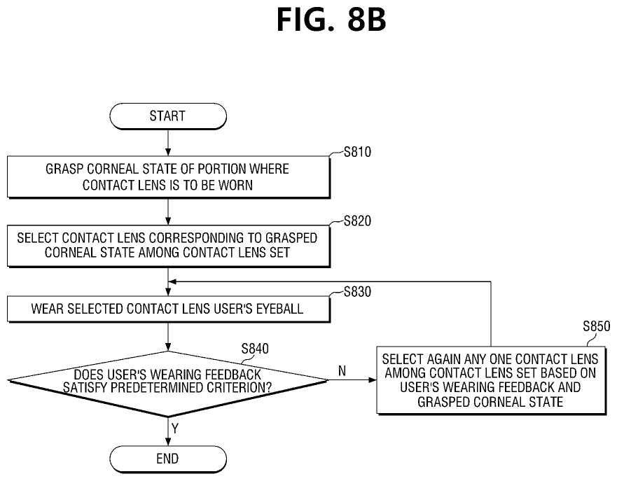

FIGS. 8A and 8B are flowcharts showing a method for wearing the contact lens including the empty space in the centric part basically and the eccentric part optionally includes an empty space in some cases according to other some embodiments.

Referring to FIG. 8A, first, it is possible to grasp the corneal state of the portion where the contact lens is to be worn (S810).

In some examples, the corneal state may include the curvature of the cornea. For example, the corneal state of the incised portion may include an average spherical curvature or an average curvature of the ellipsoid, but is not limited thereto.

In some other examples, the corneal state may include the size and diameter of the cornea, and the like.

In some other examples, the corneal state may include the amount of moisture in the cornea. For example, the corneal state may mean a numerical value of the amount of tears measured on the surface of the cornea, whether the xerophthalmia occurs, the degree thereof, and the like.

In some other examples, the corneal state may include whether the thickness is even.

For example, the corneal state may include whether a thin part and a thick part in thickness are distinguished in the user's cornea. In some other examples, the conical state may include whether eyeball surgery performed by laser-irradiating after conical incision.

However, it is of course not limited to the examples described above, and the conical state may include more various embodiments. In some examples, the conical state of the incision portion may be determined manually by a person or may be grasped using an eyeball scanner, a 3D scanner, and other medical imaging devices.

Next, a contact lens corresponding to the grasped corneal state may be selected among the contact lens set (S820).

As described above, the contact lens set may include a plurality of contact lenses having different shapes, forms, and/or materials, and the like.

In some examples, a contact lens having the highest matching rate with respect to the grasped shape and form of the cornea, and the like may be selected among the contact lens set. In this case, the matching rate may be determined manually by a person or may be determined through a device, medical device, scanning device, and the like utilizing a predetermined program.

In some other examples, a contact lens may be selected among the contact lens set, which is most suitable for the grasped amount of moisture of the cornea (for example: considering the interval between the protrusions, the protrusion height of the protrusion, and the like).

In some other examples, in a case where the eyeball surgery including a user's corneal incision process is performed, a contact lens having a high curvature or a small diameter may be selected among the contact lens set.

In some other examples, in a case where it is necessary to improve the curvature of the cornea, the curvature, and the like with a spherical shape as a result of grasping the curvature of the user's cornea and the curvature, a contact lens formed of a material with high rigidity may be selected.

That is, by selecting a contact lens corresponding to the grasped corneal state (for example: the curvature, size, moisture content, whether the corneal thickness of the cornea is even, whether predetermined surgery is performed, whether curvature improvement is required of the user's cornea, and the like) among the above-described contact lens set, it is possible to wear contact lenses optimized for the user's cornea state.

At this time, by wearing the contact lens optimized for the user's corneal state, it is possible to effectively prevent and treat the xerophthalmia, or to more firmly block the deformation of the cornea due to the intraocular pressure and thereby it has remarkable effects that the shape of the cornea can be wrapped and maintained, and/or the cornea is easily fixed after eyeball surgery.

Then, the selected contact lens may be worn on the user's eyeball (S830). Since step S830 may mean the same step as step S720 of FIG. 7, the description thereof is omitted here.

Referring to FIG. 8B, first, it is possible to grasp the corneal state of a portion where the contact lens is to be worn (S810). The step S810 has been described above with reference to FIG. 8A and thus will be omitted here.

Next, a contact lens corresponding to the grasped corneal state may be selected among the contact lens set (S820). The step S820 has been described above with reference to FIG. 8A and thus will be omitted here.

Then, the selected contact lens may be worn on the user's eyeball (S830). The step S830 has been described above with reference to FIG. 8A and thus will be omitted herein.

Then, it may be determined whether the user's wearing feedback satisfies a predetermined criterion (S840).

In some examples, the user's wearing feedback may include the presence or absence of a contact lens replacement request by the user, and the wearing suitability quantified through a predetermined algorithm. In this case, the wearing suitability may be determined based on whether a foreign body sensation is received from the user, the degree of the foreign body sensation, and/or the rate of tears flowing from the corneal centric part to the corneal periphery measured after the contact lens is worn.

In some examples, in a case where a contact lens replacement request by the user does not exist and/or the wearing suitability is greater than or equal to a predetermined value, it may be determined that the wearing feedback satisfies a predetermined criterion. Conversely, in a case where the contact lens replacement request by the user exists and/or the wearing suitability is less than the predetermined value, it may be determined that the wearing feedback does not satisfy the predetermined criterion.

However, it goes without saying that the method for determining whether the user's wearing feedback is equal to or greater than the reference value is not limited to the example described above.

Then, based on the user's wearing feedback and the grasped corneal state, any one contact lens among the contact lens set may be selected again (S850).

In some examples, in a case where the user's foreign body sensation is present or the degree of the foreign body sensation is greater than or equal to a predetermined value, a contact lens that is thinner in thickness than that of the previously selected contact lens can be selected again among the contact lenses corresponding to the grasped user's conical state (for example: curvature and size).

In some other examples, in a case where the rate of tears flowing from the conical centric part to the corneal periphery is rather fast after the user wears the contact lens, a contact lens with lower protrusions and/or smaller interval between the protrusions than those of the previously selected contact lens can be selected again among the contact lenses corresponding to the grasped user's corneal state (for example: curvature and size).

Subsequently, after step S850, steps S830 and S840 may be performed again.

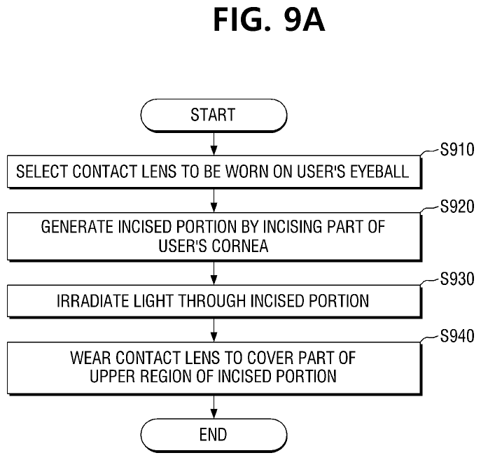

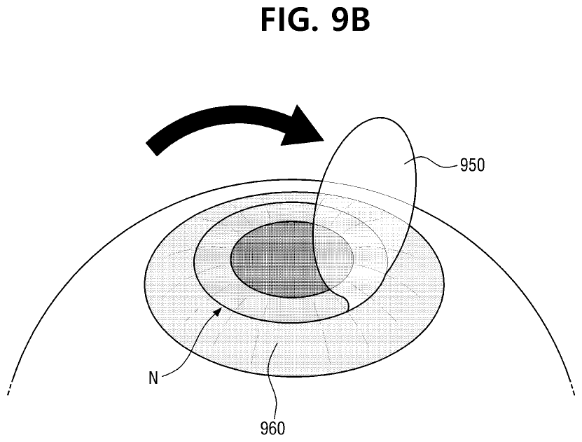

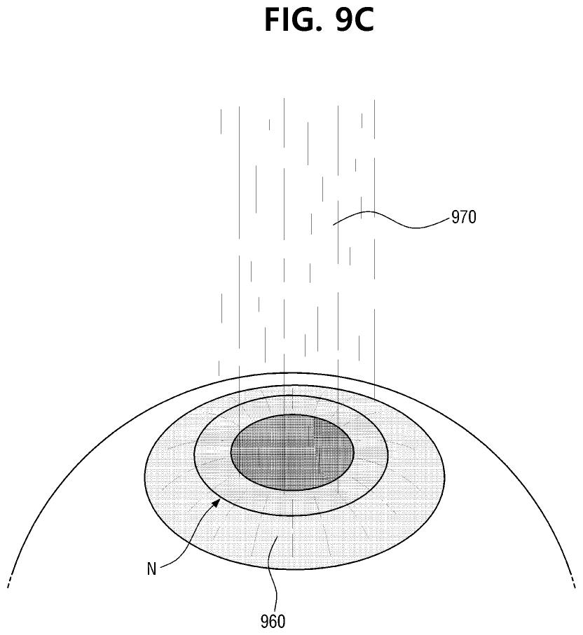

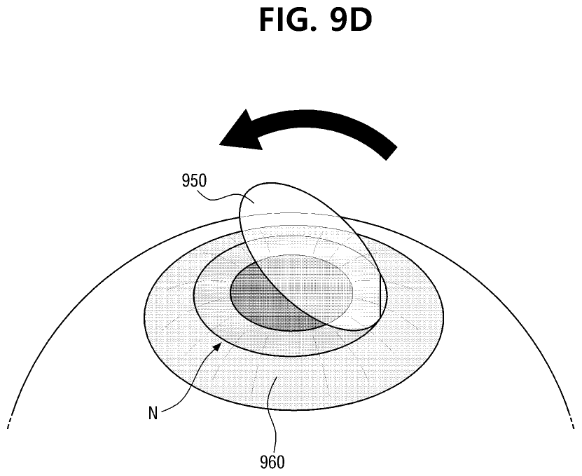

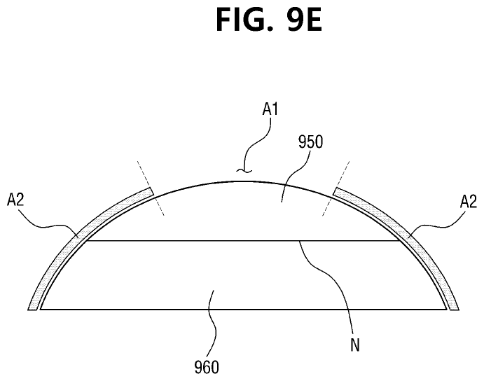

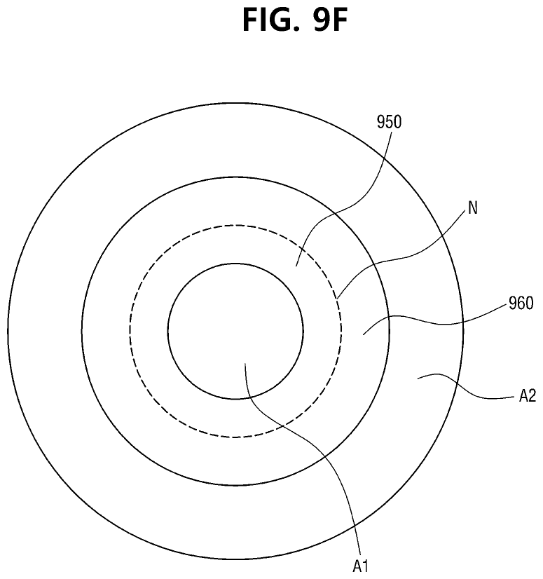

FIGS. 9A to 9F are flowcharts showing the method for wearing the contact lens including the empty space in the centric part basically and the eccentric part optionally includes an empty space in some cases, and a schematic diagrams for explaining the same, according to some other embodiment.

Referring to FIG. 9A, first, the contact lens to be worn on the user's eyeball may be selected (S910). Since step S910 may mean the same step as step S710 of FIG. 7, the description thereof is omitted here.

Then, the incised portion may be generated by incising a part of the user's cornea (S920). In this case, the cornea may mean a part of corneal epithelium, Bowman's membrane, conical stroma, posterior border plate (Desme's membrane), and conical endothelium.

In some examples, referring to FIG. 9B, an incised portion N may be generated by cutting and incising a part of the user's cornea in the horizontal direction. In this case, the user's cornea may be divided into an upper region 950 and a lower region 960, and the upper region 950 may be turned sideways.

In some examples, a part of the cornea may be incised using a laser (for example: femtosecond laser), a microkeratome (for example: microkeratome), or the like, but is not limited thereto.

Then, light may be irradiated through the incised portion (S930). Referring to FIG. 9C, in some examples, the refractive error of the cornea may be controlled by irradiating the light 1270 through the incised portion N.

Then, the contact lens may be worn to cover a part of the upper region of the incised portion (S940).

Referring to FIG. 9D, step S940 may include a process of covering the upper region 950 that has been turned over during step S930 to its original state.

FIG. 9E is a schematic diagram of a contact lens worn to cover a part of the upper region 950 of the incised portion N. FIG. 9F is a top view of FIG. 9E.

FIGS. 9E and 9F show the upper region 950 and the lower region 960 of the cornea divided based on the centric part A1, the eccentric part A2, and the incised portion N of the cover part of the contact lens.

In general, after eyeball surgery performed by incising a part of the cornea, the upper region 950 of the cornea may be covered with the lower region 960 without being sutured and separated. In this case, the position of the upper region 950 may be changed by being pushed by the intraocular pressure and sensitively responding to stimulation from the outside of the eye. In addition, a space may be generated between the upper region 950 and the lower region 960, or the upper region 950 may be peeled off. In this case, as shown in FIGS. 9A to 9F, the position of the upper region 950 can be fixed by wearing the contact lens to cover a part of the upper region 950. In some examples, such effects may be further maximized in a case where the contact lens with high curvature and small diameter is worn.

In addition, after eyeball surgery, the corneal ectasia may often occur in the corneal periphery due to the intraocular pressure (for example: 101 in FIG. 1B). In this case, in a case where the contact lens of some embodiments of the present invention is worn, such a phenomenon can be prevented.

The steps of wearing the contact lens including the empty space in the centric part basically and the eccentric part optionally includes an empty space in some cases in FIGS. 7 to 9F may be performed by a person and/or may be performed by an automated medical device, an eyeball surgery device, a surgical robot, or the like.

While the present inventive concept has been particularly shown and described with reference to exemplary embodiments thereof, it will be understood by those of ordinary skill in the art that various changes in form and details may be made therein without departing from the spirit and scope of the present inventive concept as defined by the following claims. It is therefore desired that the present embodiments be considered in all respects as illustrative and not restrictive, reference being made to the appended claims rather than the foregoing description to indicate the scope of the disclosure.

What is claimed is:

1. A contact lens comprising:
a centric part; and
a surrounding part,
wherein the centric part is empty, and only the surrounding part is worn on a user's eyeball.

2. The contact lens according to claim 1,
wherein the centric part is a region of a circle or an ellipse having a predetermined distance from a center point of the contact lens,
wherein the surrounding part is a region excluding the centric part of the contact lens.

3. The contact lens according to claim 1, wherein the contact lens, when worn on the user's eyeball, does not have a vision correction function, and when worn on the user's eyeball, stores and maintains tears in a centric area of the user's eyeball.

4. The contact lens according to claim 1, wherein the surrounding part of the contact lens comprises an empty space for the purpose of treating dry eye syndrome generated in a surrounding area of the user's eyeball.

5. The contact lens according to claim 1, wherein the surrounding part of the contact lens comprises a plurality of protrusions or grooves formed on a surface in contact with the user's eyeball.

6. The contact lens according to claim 1, wherein the surrounding part has a toric form of a predetermined size.

7. The contact lens according to claim 1, wherein a material of the surrounding part is at least one of silicone hydrogel, HAMA hydrogel, Rigid Gas Permeable (RGP), polymethyl methacrylate (PMMA), Polyvinyl Alcohol (PVA), hypergel, and cellulose acetate butylate (CAB).

8. The contact lens according to claim 1, wherein the surrounding part of the contact lens is an integral type or a combined type of a skeleton part and a cover part surrounding the skeleton part.

9. The contact lens according to claim 8, wherein the skeleton part comprises a ring assembly comprising a plurality of rings of different sizes with same center, and a connection member for connecting between the plurality of rings.

10. The contact lens according to claim 9, wherein the plurality of rings have shapes having concentric circle shapes having different sizes, or ellipse shapes having the same eccentricity and different sizes from each other.

11. The contact lens according to claim 9, wherein each of the plurality of connection members is disposed to be spaced apart from an adjacent connection member by the same angle with respect to a center of the ring assembly.

12. The contact lens according to claim 9, further comprising:
an auxiliary connection member connecting between the plurality of rings and disposed where the connection member is not disposed.

13. The contact lens according to claim 9, wherein a material of the ring is at least one of polymethyleth acrylate (PMMA), gold, alloy, platinum, titanium, and stainless steel.

14. The contact lens according to claim 8, the skeleton part comprising:
a ring assembly comprising a plurality of rings having the same center, wherein the plurality of rings have the shape of concentric circles with different sizes, and
a plurality of connection members for connecting between the plurality of rings, wherein each of the plurality of connection members is disposed apart from an adjacent connection member by the same angle with respect to the center.

15. The contact lens according to claim 14, the skeleton part further comprising:
an auxiliary connection member connecting between the plurality of rings and disposed where the connection member is not disposed.

16. The contact lens according to claim 14,
wherein the material of the ring is at least one of polymethylmethacrylate (PMMA), gold, alloy, platinum, titanium, and stainless steel.

17. The contact lens according to claim 8, the skeleton part comprising:
a ring assembly comprising a plurality of rings having the same center, wherein the plurality of rings have the shape of ellipses with the same eccentricity and different sizes, and
a plurality of connection members for connecting between the plurality of rings, wherein each of the plurality of connection members is disposed apart from an adjacent connection member by the same angle with respect to the center.

18. The contact lens according to claim 17, the skeleton part further comprising:
an auxiliary connection member connecting between the plurality of rings and disposed where the connection member is not disposed.

19. A method for wearing a contact lens comprising:
wearing a contact lens to the user's eyeball,
wherein the contact lens comprising a centric part and a surrounding part,
wherein the centric part is empty, and only the surrounding part is worn on the user's eyeball.

20. The method for wearing a contact lens according to claim 19,
wherein the centric part is a region of a circle or an ellipse having a predetermined distance from a center point of the contact lens,
wherein the surrounding part is a region excluding the centric part of the contact lens.

21. The method for wearing a contact lens according to claim 19, wherein the contact lens, when worn on the user's eyeball, does not have a vision correction function, and when worn on the user's eyeball, stores and maintains tears in a centric region of the user's eyeball.

22. A method for wearing a contact lens comprising:
wearing a contact lens to the user's eyeball,
wherein the contact lens comprising a centric part and a surrounding part,
wherein the centric part is empty, and only the surrounding part is worn on the user's eyeball,
wherein the surrounding part is an integral type or a combined type of a skeleton part and a cover part surrounding the skeleton part.

23. A method for wearing a contact lens comprising:
wearing a contact lens to the user's eyeball,
wherein the contact lens comprising a centric part and a surrounding part,
wherein the centric part is empty, and only the surrounding part is worn on the user's eyeball,
wherein the surrounding part of the contact lens comprises an empty space for the purpose of treating dry eye syndrome generated in a surrounding part of the user's eyeball.

24. A method for wearing a contact lens comprising:
processing a LASIK surgery by cutting an user's eyeball; and
wearing a contact lens to the user's eyeball after the LASIK surgery,
wherein the contact lens comprising a centric part and a surrounding part,
wherein the centric part is empty, and only the surrounding part is worn on the user's eyeball.

25. A method for wearing a contact lens comprising:
processing a LASIK surgery by cutting an user's eyeball; and
wearing a contact lens to the user's eyeball after the LASIK surgery,
wherein the contact lens comprising a centric part and a surrounding part,
wherein the centric part is empty, and only the surrounding part is worn on the user's eyeball,
wherein the centric part is a region of a circle or an ellipse having a predetermined distance from a center point of the contact lens,
wherein the surrounding part is a region excluding the centric part of the contact lens.

26. A method for wearing a contact lens comprising:
processing a LASIK surgery by cutting an user's eyeball; and
wearing a contact lens to the user's eyeball after the LASIK surgery,
wherein the contact lens comprising a centric part and a surrounding part,
wherein the centric part is empty, and only the surrounding part is worn on the user's eyeball,
wherein the surrounding part is an integral type or a combined type of a skeleton part and a cover part surrounding the skeleton part.

27. A method for wearing a contact lens comprising:
processing a LASIK surgery by cutting an user's eyeball; and
wearing a contact lens to the user's eyeball after the LASIK surgery,
wherein the contact lens comprising a centric part and a surrounding part,
wherein the centric part is empty, and only the surrounding part is worn on the user's eyeball,
wherein the surrounding part is an integral type or a combined type of a skeleton part and a cover part surrounding the skeleton part.
wherein the centric part is a region of a circle or an ellipse having a predetermined distance from a center point of the contact lens,
wherein the surrounding part is a region excluding the centric part of the contact lens.
